(19) United States
(12) Patent Application Publication
(10) Pub. No.: US 2010/0040664 A1
(43) Pub. Date: Feb. 18, 2010

(54) AABB-POLY(DEPSIPEPTIDE) BIODEGRADABLE POLYMERS AND METHODS OF USE

(75) Inventors: Ramaz Katsarava, Tbilisi (GE); Natia Ochkhikdze, Tbilisi (GE); David Tugushi, Tbilisi (GE); Zaza D. Gomurashvili, La Jolla, CA (US)

Correspondence Address: DLA PIPER LLP (US), 4365 EXECUTIVE DRIVE, SUITE 1100, SAN DIEGO, CA, 92121-2133, US

(73) Assignee: MediVas, LLC, San Diego, CA (US)

(21) Appl. No.: 12/540,285

(22) Filed: Aug. 12, 2009

Related U.S. Application Data
(60) Provisional application No. 61/088,678, filed on Aug. 13, 2008.

(51) Int. Cl. A61F 2/00; A61K 9/14; A61K 38/07; C07K 5/10

(52) U.S. Cl. 424/423; 424/489; 514/18; 530/330

(57) ABSTRACT

The invention provides AABB-poly(depsipeptide)s (AABB-PDPs), a class of biodegradable polymers composed of α-amino and α-hydroxy acids with material properties suitable for biomedical applications. These AABB-PDPs belong to the family of amino acid-based poly(ester amide)s (PEAs), which are characterized by the presence of alternating ester and amide functionalities. Containing four ether groups per basic unit, these polymers degrade rapidly by biotic or abiotic hydrolytic action to release dispersed bioactive agents at a controlled delivery rate, are non-toxic, produce digestible breakdown products, and are easy to fabricate by solution polycondensation.

### RELATED APPLICATIONS

This application claims the benefit of priority under 35 U.S.C. §119(e) of U.S. Ser. No. 61/088,678, filed Aug. 13, 2008.

### FIELD OF THE INVENTION

A depsipeptide (“depsi” comes from the Greek word for ester) is a chemical structure consisting of both ester and amide bonds (FIG. 1) (J. Zhang, et al. Biomacromolecules (2007) 8:3015-3024). The chemical structure of depsipeptides may appear to be “engineered” from amino acids; however, depsipeptides actually occur naturally in certain lactic acid bacteria. Moreover, depsipeptides, primarily in cyclic form, have been explored as potential anticancer agents in drug discovery.

Poly(depsipeptide)s (PDPs) represent a class of biodegradable polymers composed of α-amino and α-hydroxy acids with material properties suitable for biomedical applications. PDPs belong to the family of amino acid-based poly(ester amide)s (PEAs), which are characterized by the presence of alternating ester and amide functionalities. Several research groups have been focusing on the synthesis of polydepsipeptides (AB-PDPs) (J. Helder and Feijen J. Macromol. Chem. Rapid Comm. (1986) 7:193). These polymers have potential applications in drug delivery and tissue engineering as being degradable via hydrolytic scission into biocompatible chemicals (Ohya Y, et al. “Cell attachment and growth on films prepared from poly(depsipeptide-co-lactide) having various functional groups.” J. Biomed Mater. Res., Part A (2003) 6(1):79-88).

There are two reported synthetic approaches to AB-type polydepsipeptides: a) by solution polycondensation of corresponding di, tri, or higher depsipeptide (M. Yoshida et al. Makromol. Chem. Rapid Commun., (1990) 11:337) and b) ring opening polymerization of cyclic monomers, such as morpholine-2,5-dions,-six-membered heterocyclic compounds composed of α-hydroxy and α-amino acid (P. J. A In't Veld et al. J. Poly. Sci., Part A. Polym. Chem. (1994) 32:1063). The first way of synthesizing AB-PDPs utilizes multi-stage peptide synthesis and is complex and expensive. The second way, by melt polymerization of morpholine-2,5-dions in the presence of organotin catalyst, is more facile and less expensive but provides low yields of monomers such as morpholine-2,5-dions (max. 30% per α-amino acid) and, in some cases, forms low-molecular-weight oligomers or polymers with unfavorable mechanical properties or synthetic restrictions.

Additional members of the PEA family that have proven to be suitable materials for biomedical applications because of their excellent blood and tissue compatibily are regular AABB-type bio-analogous poly(ester amides) (AABB-PEAs), which consist of nontoxic building blocks, such as hydrophobic α-amino acids, aliphatic diols and di-carboxylic acids (K. DeFife et al. Transcatheter Cardiovascular Therapeutics—TCT2004 Conference. Poster presentation. Washington D.C. 2004). Regular AABB-PEAs also exhibit biologic degradation profiles (G. Tsitlanadze, et al. J. Biomater. Sci. Polymer Edn. (2004) 15:1-24). The controlled biological enzymatic degradation and low nonspecific degradation rates of such PEAs make them attractive for drug delivery applications.

These properties of the bio-analogous PEAs provide advantages over widely used aliphatic polyesters, such as polylactic acid (PLA) and polyglycolic acid (PGA). Aliphatic ester-groups in macromolecules of PLA and PGA contribute to rapid hydrolytic degradation rates, but polymer surfaces of PLA and PGA are known to display poor adhesion and cell growth; whereas good adhesion and cell growth properties are considered important indicators of beneficial cell-biomaterial interactions (Cook, A D, et al. J. Biomed Mater. Res., (1997) 35:513-523).

However, not all environments in the body possess endogenous biological enzymes. Therefore, despite these advances in the art, there is need for new and better members of the PEA family of polymers that are suitable for drug delivery applications, particularly polymers that degrade rapidly by biotic or abiotic hydrolytic action to release dispersed bioactive agents at a controlled delivery rate, are non-toxic, produce digestible breakdown products, and are easy to fabricate.

### SUMMARY OF THE INVENTION

Accordingly in one embodiment, the invention provides degradable polymer compositions comprising a AABB-polydepsipeptide (AABB-PDP) having a chemical formula described by general structural formula (I),

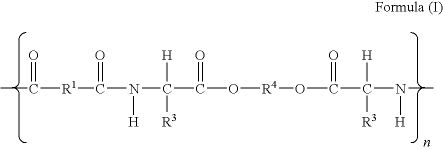

wherein n ranges from about 5 to about 150;

at least one R¹ is independently selected from residues of O,O′-diacyl-bis-(alpha hydroxy acid) of formula (III) below, wherein R⁵ is H or CH₃ and R⁶ is independently selected from (C₂-C₁₂) alkylene or (C₂-C₁₂) alkenylene and wherein additional R's can be selected from the group consisting of (C₂-C₂₀) alkylene, (C₂-C₂₀) alkenylene, (±)-bis(4-carboxyphenoxy)-(C₁-C₈) alkane, saturated or unsaturated residues of therapeutic di-acids, and combinations thereof;

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), saturated or unsaturated therapeutic diol residues, and combinations thereof;

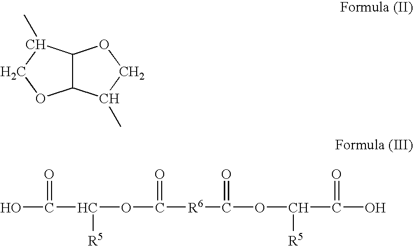

or a AABB-PDP having a chemical formula described by structural formula (IV):

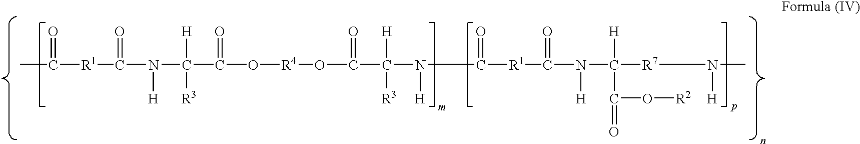

wherein n ranges from about 5 to about 150, m ranges about 0.1 to 0.9; p ranges from about 0.9 to 0.1;

at least one R¹ is independently selected from residues of O,O′-diacyl-bis-(alpha hydroxy acid) of formula (III) below, wherein in Formula (III) R⁵ is H or methyl and R⁶ is independently selected from (C₂-C₁₂) alkylene and (C₂-C₁₂) alkenylene, and additional R¹s can be selected from the group consisting of (C₂-C₂₀) alkylene, (C₂-C₂₀) alkenylene, α,ω-bis(4-carboxyphenoxy)-(C₁-C₈) alkane, saturated or unsaturated residues of therapeutic di-acids, and combinations thereof,

R² is independently selected from the group consisting of hydrogen, (C₁-C₁₂) alkyl, (C₆-C₁₀) aryl or a protecting group;

R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;

R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), residues of saturated or unsaturated therapeutic diols and combinations thereof, and

R⁷ is independently selected from the group consisting of (C₂-C₂₀) alkyl and (C₂-C₂₀) alkenyl.

In another embodiment, the invention provides surgical devices comprising the invention AABB-PDP composition in which at least one bioactive agent is disbursed. Such surgical devices include solid implants, particles, and coatings of the composition on at least a portion of the surface of a surgical device for delivery of the bioactive agent disbursed in the AABB-PDP composition.

In yet another embodiment, the invention provides methods for preparing an O,O′-diacyl-bis-(alpha hydroxy acid) having a chemical formula described by structural formula (III)

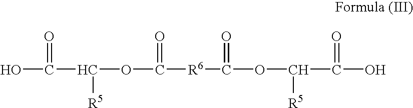

wherein R⁵ is H or CH₃ and R⁶ is an acyl independently selected from (C₂-C₁₂) alkylene and (C₂-C₁₂) alkenylene, said method comprising:

a) forming an acid di-chloride of the acyl in an organic basic solvent that acts as a hydrogen chloride acceptor and catalyst;

b) interacting the acid di-chloride with glycolic or lactic acid in dry ethyl acetate in the presence of the solvent to form solid O,O′-diacyl-bis-(alpha hydroxy acid) product; and

c) collecting solid O,O′-diacyl-bis-(alpha hydroxy acid) product formed in b) from the solvent.

In still another embodiment, the invention provides methods for delivering a bioactive agent to a subject, said method comprising administering to the subject in vivo an invention AABB-PDP composition containing the bioactive agent dispersed therein.

### A BRIEF DESCRIPTION OF THE FIGURES

FIG. 1 is a drawing describing the chemical structural formula of a (depsi-peptide). The central part shown is referred to as a lactide or glycolide residue.

FIG. 2 is a scan showing an FR-IR spectrum in KBr of O,O′-adipoyl-bis-(glycolic acid) (Compound 1.1).

FIG. 3 is a scan showing a 300 MHz ¹H NMR spectrum of diester diacid (compound 1.1) in d6-DMSO/CCl₄ (1:3 v/v) mixture.

FIG. 4 is a scan showing an FTIR spectrum in KBr of active di-p-nitrophenyl ester of O,O′-adipoyl-bis-glycolic acid (Compound 2.1).

FIG. 5 is a scan showing a 300 MHz ¹H NMR spectrum of active diester (compound 2.1) in d6-DMSO/CCl₄ (1:3 v/v) mixture).

FIG. 6 is a scan showing an FTIR spectrum of a PDP 4-GA-Phe-8 film from CHCl₃ solution on KBr plate.

FIG. 7 is a scan showing a 300 MHz ¹H-NMR spectrum of PDP 4-GA-Phe-8 in DMSO-d₆/CCl₄.

FIGS. 8A and 8B are scans showing differential scanning calorimeter (DSC) thermograms (data based on two scans each) of invention AABB-PDP samples 4-GA-Leu-12 (FIG. 8A) and 4-GA-Phe-8 (FIG. 8B).

FIG. 9 is a bar graph showing hydrolysis rates (from potentiometric titration data) of AABB-PDP 4-GA-Leu-12-gray bars and regular PEA polymer 8-Leu-6, white bars at various pH values. A potentiometric titrator and 0.02 N NaOH water solution were used for automatic titration of carboxyl groups released after hydrolysis of ester bonds.

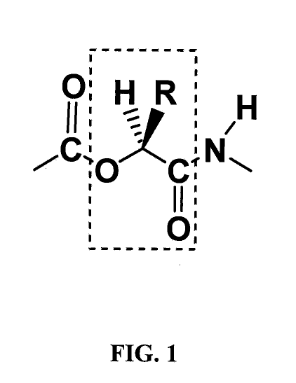

### A DETAILED DESCRIPTION OF THE INVENTION

The present invention is based on the discovery of new type of aliphatic AABB-poly(depsipeptide) (AABB-PDP) polymer composition with significant improvement in hydrolytic degradation rates as compared to those of regular aliphatic di-acid-containing AABB-PEA polymers. The invention AABB-PDPs are synthesized by polycondensation of active di-p-nitrophenyl esters of O,O′-diacyl-bis-(alpha hydroxy acids) with di-p-toluenesulfonic acid salts of bis-(α-amino acid)-α,ω-alkylene diesters.

Bis(α-amino acid)-α,ω-alkylene-diester is a type of diamine monomer that is useful for active polycondensation (APC), and which inherently contains two aliphatic ester linkages. Such ester groups can be enzymatically recognized by various esterases, including biological esterases. Condensation of diamine monomers, for example, with activated aliphatic di-acid esters, results in a regular AABB-PEA macromolecule with ester and amide functionalities as well as alkylene chains in the backbone of the elemental chain unit.

By contrast, the di-acid-type compounds used in synthesis of the invention AABB-PDP compositions include at least one non-toxic fatty aliphatic homolog, an O,O′-diacyl-bis-(alpha hydroxy acids) of formula (III) below, which is composed of residues of short aliphatic non-toxic di-acids and glycolic or lactic acids. These di-acid type compounds also inherently contain two-ester groups that easily can be cleaved by both biotic (enzymatic) and abiotic hydrolysis. Therefore, the invention AABB-PDP compositions (Formulas I and IV below) possess an increased number of ester groups—a total of four—in the polymer elemental chain unit as compared with previously known PEA polymers. These additional ester groups confer more rapid biodegradability than that of PEA polymers composed of residues of aliphatic di-acids. Additionally, the invention AABB-PDP compositions, particles and coatings thereof can be digested by abiotic (chemical) hydrolysis.

The invention AABB-PDP polymer compositions of Formula (IV) below can include a second amino acid-based monomer residue, such as a C-protected L-lysine-based monomer, which contributes an additional aliphatic residue to the monomer backbone to introduce additional chain flexibility into the polymer.

Accordingly in one embodiment, the invention provides biodegradable polymer compositions comprising a AABB-polydepsipeptide (AABB-PDP) having a chemical formula described by general structural formula (I),

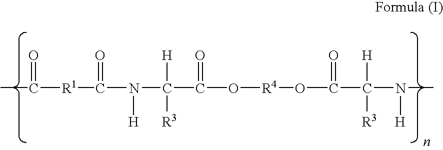

wherein n ranges from about 5 to about 150;

at least one R¹ is independently selected from residues of O,O′-diacyl-bis-(alpha hydroxy acid) of formula (III) below, wherein in Formula (III) R⁵ is H or methyl and R⁶ is independently selected from (C₂-C₁₂) alkylene or (C₂-C₁₂) alkenylene and additional R¹s can be selected from the group consisting of (C₂-C₂₀) alkylene, (C₂-C₂₀) alkenylene, α,ω-bis(4-carboxyphenoxy)-(C₁-C₈) alkane, saturated or unsaturated residues of therapeutic di-acids, and combinations thereof;

R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), saturated or unsaturated therapeutic diol residues, and combinations thereof;

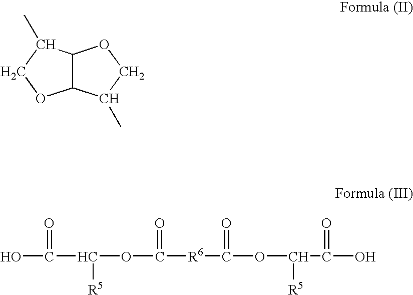

or a AABB-PDP having a chemical formula described by structural formula (IV):

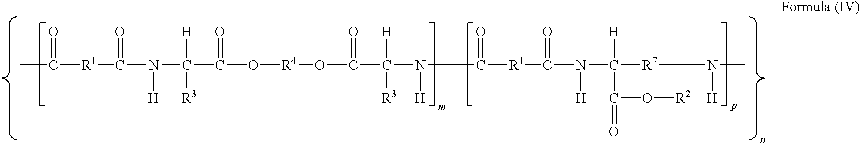

wherein n ranges from about 5 to about 150, m ranges about 0.1 to 0.9; p ranges from about 0.9 to 0.1;

at least one R¹ is independently selected from residues of O,O′-diacyl-bis-(alpha hydroxy acid) of formula (III) below, wherein in Formula (III) R⁵ is H or methyl and R⁶ is independently selected from (C₂-C₁₂) alkylene and (C₂-C₁₂) alkenylene, and additional R¹s can be selected from the group consisting of (C₂-C₂₀) alkylene, (C₂-C₂₀) alkenylene, α,ω-bis(4-carboxyphenoxy)-(C₁-C₈) alkane, saturated or unsaturated residues of therapeutic di-acids, and combinations thereof,

R² is independently selected from the group consisting of hydrogen, (C₁-C₁₂) alkyl, (C₆-C₁₀) aryl or a protecting group;

R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;

R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), residues of saturated or unsaturated therapeutic diols and combinations thereof, and

R⁷ is independently selected from the group consisting of (C₂-C₂₀) alkyl and (C₂-C₂₀) alkenyl.

For example in one embodiment, in formula (III), R⁵ is selected from H (as in a residue of glycolic acid) and CH₃ (as in D-, L- or D, L-lactide).

In another embodiment, R⁶ is independently selected from the group consisting of (CH₂)₄, (CH₂)₆, and (CH₂)₈.

In yet another embodiment, R⁷ is independently selected from the group consisting of (C₃-C₆) alkyl and (C₃-C₆) alkenyl, preferably —(CH₂)₄—.

The AABB-PDP polymers in the invention compositions are poly-condensates. The ratios “m” and “p” in Formula (IV) are defined as irrational numbers in the description of these poly-condensate polymers. Moreover, as “m” and “p” will each take up a range within any poly-condensate, such a range cannot be defined by a pair of integers. Each polymer chain is a string of monomer residues linked together by the rule that all bis-amino acid diol (i) and a directional amino acid (e.g. lysine) monomer residues (ii) are linked either to themselves or to each other by a diacid monomer residue (iii). Thus, only linear combinations of i-iii-i; i-iii-ii (or ii-iii-i) and ii-iii-ii are formed. In turn, each of these combinations is linked either to themselves or to each other by a diacid monomer residue (iii). Each polymer chain is therefore a statistical, but non-random, string of monomer residues composed of integer numbers of monomers, i, ii and iii. However, in general for polymer chains of any practical average molecular weight (i.e., sufficient mean length), the ratios of monomer residues “m” and “p” in formula (IV) will not be whole numbers (rational integers). Furthermore, for the condensate of all poly-dispersed copolymer chains, the numbers of monomers i, ii and iii averaged over all of the chains (i.e. normalized to the average chain length) will not be integers. It follows that the ratios can only take irrational values (i.e., any real number that is not a rational number). Irrational numbers, as the term is used herein, are derived from ratios that are not of the form n/j, where n and j are integers.

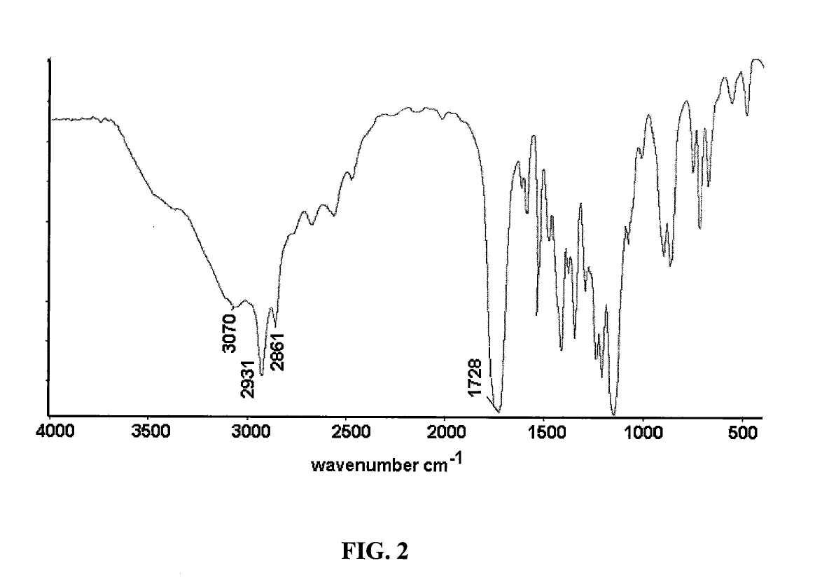

As used herein, the terms “amino acid” and “α-amino acid” mean a chemical compound containing an amino group, a carboxyl group and an R group, usually pendant, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof. However, it has to be noted that the orientation of in AABB-PDPs is a directional (head-to-head as is shown by arrows in FIG. 2); whereas in known AB-PDPs the orientation of the α-amino acids in the polymer backbone is directional (conventional, head-to-tail). Hence, it is expected that the invention AABB-PDPs will show lower immunogenicity than AB-PDPs.

In addition in the “p” monomer of Formula (IV), an unconventional amino acid is formed in which the aliphatic moiety R⁷ is inserted within the polymer backbone to provide additional flexibility to the polymer while optionally providing a functionality in the pendant group, such as a carboxyl group (when R² is H).

As used herein the term “bioactive agent” encompasses therapeutic diols or di-acids incorporated into the polymer backbone of an invention composition as well a bioactive agent as disclosed herein that is dispersed in the polymer of the invention composition. As used herein, the term “dispersed” is used to refer to a bioactive agent that is mixed into, dissolved in, homogenized with, and/or covalently bound to an invention AABB-PDP polymer, for example, attached to a functional group in the polymer of an invention composition or to the surface of a polymer particle. Such bioactive agents may include, without limitation, small molecule drugs, peptides, proteins, DNA, cDNA, RNA, sugars, lipids and whole cells. The bioactive agents can be administered in polymer particles having a variety of sizes and structures suitable to meet differing therapeutic goals and routes of administration.

The term, “biodegradable” as used herein to describe the invention AABB-PDP compositions means the polymer used therein is capable of being broken down into innocuous products in the normal functioning of the body due to abiotic (chemical) and biotic enzymatic processes. The invention AABB-PDP polymers show a high rate of nonspecific chemical hydrolysis due to the presence in the invention polymers of polarized ester bonds formed by glycolic or adipic acid residues. This characteristic is believed to be important for degradation of devices implanted in in vivo body sites, such as the blood stream, where the concentration of bioenzymes (e.g., proteases and esterases) is negligible. By contrast, amide linkages in regular PEA polymers require catalytic action of bioenzymes-acylases—for rapid scission of amide bonds.

Optionally, the amino termini of the polymers can be acetylated or otherwise capped by conjugation to any other acid-containing, biocompatible molecule, to include without restriction organic acids, bioinactive biologics, and bioactive agents as described herein. In one embodiment, the entire polymer composition, and any particles made thereof, is substantially biodegradable.

In one alternative, at least one α-amino acids is used in fabrication of the invention polymers is a biological α-amino acid. For example, when the R³s are CH₂Ph, the biological α-amino acid used in synthesis is L-phenylalanine. In alternatives wherein the R³s are CH₂CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃) CH₂CH₃), phenylalanine (when the R³s are CH₂C₆H₅), or methionine (when the R³s are —(CH₂)₂SCH₃), and combinations thereof. In yet another alternative embodiment, all of the various α-amino acids contained in the invention AABB-PDP compositions are biological α-amino acids, as described herein.

In another alternative, all of the R's are independently selected from residues of O,O′-diacyl-bis-(alpha hydroxy acid) of formula (III) above, wherein in R⁵ is H or methyl and R⁶ is independently selected from (C₂-C₁₂) alkylene or (C₂-C₁₂) alkenylene.

In yet another embodiment, it is presently preferred that R⁶ is independently selected from (C₂-C₄) alkylene or (C₂-C₄) alkenylene.

In still another embodiment, particles of the invention AABB-PDP polymer compositions are sized to agglomerate in vivo forming a time-release polymer depot for local delivery of bioactive agents dispersed therein to surrounding tissue/cells when injected in vivo. Methods for fabrication of particles from PEA polymers are well known in the art and described, for example, in US Patent Publication No. 20060177416.

The invention AABB-PDP compositions can be formulated to provide a variety of properties, including but not limited to, a desired controlled rate of degradation or propensity for cell adhesion, by selection of the building blocks incorporated therein as described herein. For example, according to the synthetic methods described herein, functional AABB-PDPs to which a chemical moiety can be attached can be synthesized by incorporating into the polymeric backbones either moieties that provide free functional groups (for example, lysine, glutamic acid, or 1,3-diamino-2-hydroxy propane) or unsaturated moieties (for example, active fumarates or monomers described by Formula (III) wherein the diol used is unsaturated).

As used herein, a “therapeutic diol or di-acid” means any diol or di-acid molecule, whether synthetically produced, or naturally occurring (e.g., endogenously) that affects a biological process in a mammalian individual, such as a human, in a therapeutic or palliative manner when administered to the mammal.

As used herein, the term “residue of a therapeutic di-acid” means a portion of such a therapeutic di-acid that excludes the two carboxyl groups of the di-acid. As used herein, the term “residue of a therapeutic diol” means a portion of a therapeutic diolthat excludes the two hydroxyl groups of the diol. The corresponding therapeutic di-acid or diol containing the “residue” thereof is used in synthesis of the polymer compositions. The residue of the therapeutic di-acid or diol is incorporated into the polymer backbone and reconstituted in vivo (or under similar conditions of pH, aqueous media, and the like) to the corresponding diol or di-acid upon release in a controlled manner from the backbone of the polymer by biodegradation. The release rate of the di-acid or diol depends upon the degradation properties of the particular AABB-PDP of the composition, and the enzymes, biotic and/or abiotic, present at the particular in vivo site of implant, as described herein.

As used herein the term “bioactive agent” means a bioactive agent as disclosed herein that is not incorporated into the polymer backbone. One or more such bioactive agents may optionally be dispersed in the invention AABB-PDP compositions. As used herein, the term “dispersed” is used to refer to bioactive agents not incorporated into the polymer backbone and means that the bioactive agent is mixed, dissolved, homogenized with, and/or covalently bound to the AABB-PDP polymer in the invention composition. For example the bioactive agent can be attached to a functional group in the polymer of the composition or to the surface of a polymer particle or coating on a particle or medical device. To distinguish backbone-incorporated therapeutic diols and di-acids from those that are not incorporated into the polymer backbone, (as a residue thereof), such dispersed therapeutic diols and di-acids are referred to herein as “bioactive agent(s)” and may be contained within polymer conjugates or otherwise dispersed in the polymer composition in the same manner as other bioactive agents, as described below.

The term, “degradable” as used herein to describe the polymers used in the invention AABB-PDP compositions means the polymer is capable of being broken down by enzymatic hydrolysis into innocuous products in the normal functioning of the body. As illustrated in Example 4 herein, cleavage of ester bonds (4 per molecule) easily forms readily digestible breakdown products: 2 moles of depsipeptide and one mole of di-acid. In the case of a naturally occurring therapeutic di-acid in the polymer backbone, the breakdown products will further include the reconstituted di-acid and/or diol.

The AABB-PDP polymers in the invention compositions are typically chain terminated with amino groups. Optionally, these amino termini can be acetylated or otherwise capped by conjugation to any other acid-containing, biocompatible molecule, to include without restriction organic acids, bioinactive biologics, and bioactive agents as described herein. In one embodiment, the entire AABB-PDP composition is biodegradable, for example by bioenzymes.

Although each invention AABB-PDP is fabricated using at least one active di-p-nitrophenyl ester of O,O′-diacyl-bis-(alpha hydroxy acids), optionally, therapeutic diol compounds also can be used to prepare bis(α-amino acid) diesters of therapeutic diol monomers, or bis(carbonate) of therapeutic di-acid monomers, for introduction into the backbone of invention AABB-PDPs. Included in such therapeutic diols are naturally occurring therapeutic diols, such as 17-β-estradiol, a natural and endogenous hormone, useful in preventing restenosis in arteries and tumor growth (Yang, N. N., et al. Science (1996) 273:1222-1225; S. Parangi et al Cancer Res. (1997) 57:81-86; and T. Fotsis Nature (1994) 368:237-239). The safety profiles of such endogenously occurring therapeutic diol molecules are believed to be superior to those of synthetic and/or non-endogenous molecules having a similar utility, such as sirolimus.

When the invention AABB-PDP polymer containing a residue of 17-β-estradiol is used to fabricate particles and the particles are implanted into a patient, for example, following percutaneous transluminal coronary angioplasty (PTCA), 17-β-estradiol released from the particles in vivo can help to prevent post-implant restenosis in the patient. 17-β-estradiol, however, is only one example of a diol with therapeutic properties that can be incorporated into the backbone of a AABB-PDP polymer in accordance with the invention. In one aspect, any bioactive steroid-diol containing primary, secondary or phenolic hydroxyls can be used for this purpose. Many steroid esters that can be made from bioactive steroid diols for use in the invention are disclosed in European application EP 0127 829 A2.

In addition, synthetic steroid diols based on testosterone or cholesterol, such as 4-androstene-3,17 diol (4-Androstenediol), 5-androstene-3,17 diol (5-Androstenediol), 19-nor5-androstene-3,17 diol (19-Norandrostenediol) are suitable for incorporation into the backbone of AABB-PDP polymers according to this invention. Moreover, therapeutic diol compounds suitable for use in preparation of the invention AABB-PDP compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycinA; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

Suitable naturally occurring and synthetic therapeutic di-acids that can be used to prepare an amide linkage in the PEA polymer compositions of the invention include, for example, bambermycin(s); benazepril; carbenicillin; carzinophillin A; cefixime; cefininox cefpimizole; cefodizime; cefonicid; ceforanide; cefotetan; ceftazidime; ceftibuten; cephalosporin C; cilastatin; denopterin; edatrexate; enalapril; lisinopril; methotrexate; moxalactam; nifedipine; olsalazine; penicillin N; ramipril; quinacillin; quinapril; temocillin; ticarcillin; Tomudex® (N-[[5-[[(1,4-Dihydro-2-methyl-4-oxo-6-quinazolinyl)methyl]methylamino]-2-thienyl]carbonyl]-L-glutamic acid), and the like. The safety profile of naturally occurring therapeutic di-acids is believed to surpass that of synthetic therapeutic di-acids. The therapeutic di-acid can be either a saturated or an unsaturated di-acid.

The chemical and therapeutic properties of the above described therapeutic diols and di-acids as tumor inhibitors, cytotoxic antimetabolites, antibiotics, and the like, are well known in the art and detailed descriptions thereof can be found, for example, in the 13th Edition of The Merck Index (Whitehouse Station, N.J., USA).

The biodegradable AABB-PDP polymers can contain from one to multiple different α-amino acids per polymer molecule and preferably have weight average molecular weights ranging from about 20,000 Da to about 80,000 Da. In particular the polymers whose fabrication is described in the Examples herein range in molecular weight from about 35,000 Da to 46,000 Da, with M_{w}/Mₙ—from 1.36 to 1.46.

In yet another embodiment, the invention provides methods for delivering one or more bioactive agents to a local site in the body in a subject in a controlled manner. In this embodiment, the invention methods involve injecting into a site in the body of the subject an invention AABB-PDP that has been formulated as a dispersion of polymer particles with at least one bioactive agent dispersed therein. The injected particles agglomerate to form a polymer depot of particles of increased size and the agglomeration will slowly release the individual particles, which will degrade by enzymatic action to release the dispersed bioactive agent(s) in vivo in a controlled manner over a period from about one week to about six months.

A dispersion of particles of the invention AABB-PDP polymers can be injected, for example subcutaneously, intramuscularly, or into an interior body site, such as an organ. Polymer particles of sizes capable of passing through pharmaceutical syringe needles ranging in size from about 19 to about 27 Gauge, for example those having an average diameter in the range from about 1 μm to about 200 μm, can be injected into an interior body site, and will agglomerate to form particles of increased size that form a depot to dispense the dispersed bioactive agent(s) locally. In other embodiments, the biodegradable polymer particles act as a carrier for the bioactive agent into the circulation for targeted and timed release systemically. Invention polymer particles in the size range of about 10 nm to about 500 rm will enter directly into the circulation for such purposes.

While the bioactive agent(s) can be dispersed within the polymer matrix without chemical linkage to the polymer carrier, it is also contemplated that one or more bioactive agents or covering molecules can be covalently bound to the biodegradable polymers via a wide variety of suitable functional groups. For example, a free carboxyl group can be used to react with a complimentary moiety on a bioactive agent or covering molecule, such as a hydroxy, amino, or thio group, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, one or more bioactive agent can be linked to any of the polymers of structures (I) and (IV) through an amide, ester, ether, amino, ketone, thioether, sulfinyl, sulfonyl, or disulfide linkage. Such a linkage can be formed from suitably functionalized starting materials using synthetic procedures that are known in the art.

For example, in one embodiment a polymer can be linked to a bioactive agent via a free carboxyl group (e.g., COOH) of the polymer. Specifically, an invention AABB-PDP composition of structural formula (I) or (IV) can react with an amino functional group or a hydroxyl functional group of a bioactive agent to provide a biodegradable polymer having the bioactive agent attached via an amide linkage or ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Water soluble covering molecule(s), such as poly(ethylene glycol) (PEG); phosphatidylcholine (PC); glycosaminoglycans including heparin; polysaccharides including chitosan, alginates and polysialic acid; poly(ionizable or polar amino acids) including polyserine, polyglutamic acid, polyaspartic acid, polylysine and polyarginine; as described herein, and targeting molecules, such as antibodies, antigens and ligands, are bioactive agents that can also be conjugated to the polymer on the exterior of particles formed from the AABB-PDP composition after production of the particles to block active sites not occupied by a bioactive agent or to target delivery of the particles to a specific body site as is known in the art. The molecular weights of PEG molecules on a single particle can be substantially any molecular weight in the range from about 200 to about 200,000, so that the molecular weights of the various PEG molecules attached to the particle can be varied.

Alternatively, a bioactive agent or covering molecule can be attached to the polymer via a linker molecule or by cross-linking two or more molecules of the polymer as described herein. Indeed, to improve surface hydrophobicity of the biodegradable polymer, to improve accessibility of the biodegradable polymer towards enzyme activation, and to improve the release profile of the bioactive agents from the biodegradable polymer, a linker may be utilized to indirectly attach a bioactive agent to the biodegradable polymer. In certain embodiments, the linker compounds include poly(ethylene glycol) having a molecular weight (M_{W}) of about 44 to about 10,000, preferably 44 to 2000; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄) alkyl, (C₂-C₂₄) alkenyl, (C₂-C₂₄) alkynyl, (C₂-C₂₀) alkyloxy, (C₃-C₈) cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆) alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein used to describe the above linkers, “alkenyl” refers to straight or branched chain hydrocarbyl groups having one or more carbon-carbon double bonds.

As used herein used to describe the above linkers, “alkynyl” refers to straight or branched chain hydrocarbyl groups having at least one carbon-carbon triple bond.

As used herein used to describe the above linkers, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, poly-L-lysine-L-tyrosine, and the like.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a bioactive agent is a polypeptide presented as a retro-inverso or partial retro-inverso peptide.

In other embodiments, a bioactive agent may be mixed with a photocrosslinkable version of the polymer in a matrix, and, after crosslinking, the material is dispersed (ground) to form particles having an average diameter in the range from about 0.1 to about 10 μm.

The linker can be attached first to the polymer or to the bioactive agent or covering molecule. During synthesis, the linker can be either in unprotected form or protected from, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenation for saturated polymer backbones, mild acid or base hydrolysis for unsaturated polymers, or any other common de-protection method that is known in the art. The de-protected linker can then be attached to the bioactive agent or covering molecule, or to the polymer

Polymer—Bioactive agent Linkage

In one embodiment, the polymers used to make the invention AABB-PDP compositions as described herein have one or more bioactive agent directly linked to the polymer. The residues of the polymer can be linked to the residues of the one or more bioactive agents. For example, one residue of the polymer can be directly linked to one residue of a bioactive agent. The polymer and the bioactive agent can each have one open valence. Alternatively, more than one bioactive agent, multiple bioactive agents, or a mixture of bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer having at least one diol or di-acid bioactive agent incorporated into the backbone of the polymer.

As used herein, a “residue of a polymer” refers to a radical of a polymer having one or more open valences. Any synthetically feasible atom, atoms, or functional group of the polymer (e.g., on the polymer backbone or pendant group) is substantially retained when the radical is attached to a residue of a bioactive agent. Additionally, any synthetically feasible functional group (e.g., carboxyl) can be created on the polymer (e.g., on the polymer backbone as a pendant group or as chain termini) to provide the open valence, provided bioactivity of the backbone therapeutic agent is substantially retained when the radical is attached to a residue of a bioactive agent. Based on the linkage that is desired, those skilled in the art can select suitably functionalized starting materials that can be used to derivatize the polymers used in the present invention using procedures that are known in the art.

As used herein, a “residue of a compound of structural formula (*)” refers to a radical of an AABB-PDP composition of structural formula (I) or (IV) as described herein having one or more open valences. Any synthetically feasible atom, atoms, or functional group of the compound (e.g., on the polymer backbone, pendant or end group) can be removed to provide the open valence, provided bioactivity of the backbone therapeutic agent is substantially retained when the radical is attached. Additionally, any synthetically feasible functional group (e.g., carboxyl) can be created on the compound of formulas (I) and (IV) (e.g., on the polymer backbone or pendant group) to provide the open valence, provided bioactivity of the backbone therapeutic agent is substantially retained when the radical is attached to a residue of a bioactive agent. Based on the linkage that is desired, those skilled in the art can select suitably functionalized starting materials that can be used to derivative the AABB-PDP compositions of formulas (I) and (IV) using procedures that are known in the art.

For example, the residue of a bioactive agent can be linked to the residue of a an AABB-PDP composition of structural formula (I) or (IV) through an amide (e.g., —N(R)C(═O)— or C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆) alkyl. Such a linkage can be formed from suitably functionalized starting materials using synthetic procedures that are known in the art. Based on the linkage that is desired, those skilled in the art can select suitably functional starting material to derivatize any residue of an AABB-PDP composition of structural formula (I) or (IV) and thereby conjugate a given residue of a bioactive agent using procedures that are known in the art. The residue of a bioactive agent can be linked to any synthetically feasible position on the residue of a compound of structural formula (I) or (IV). Additionally, more than one residue of a bioactive agent can be directly linked to the AABB-PDP composition.

The number of bioactive agents that can be linked to the polymer molecule can typically depend upon the molecular weight of the polymer and the number of backbone bioactive agents incorporated into the polymer. For example, for a compound of structural formula (I), wherein n is about 5 to about 150, preferably about 5 to about 70, up to about 150 bioactive agent molecules (i.e., residues thereof) can be directly linked to the polymer (i.e., residue thereof) by reacting the bioactive agent with backbone, pendant or terminal groups of the polymer. The number of sites for linkage of a bioactive agent in the invention AABB-PDP compositions is accordingly reduced by the number of backbone therapeutic diol or di-acids incorporated into the polymer. In unsaturated polymers, bioactive agents can also be reacted with double (or triple) bonds in the polymer, provided that the therapeutic diol or di-acid residues incorporated into the polymer backbone do not contain any double (or triple) bonds themselves. Hence, in the case of estradiol incorporated into the backbone, linkage of a bioactive agent at a double bond in the polymer composition would not be recommended, to prevent bonding of the bioactive agent to a double bond in the backbone diol or di-acid residue (i.e., the estradiol) in a reaction.

In the AABB-PDP composition, either in the form of particles or not, a bioactive agent can be covalently attached directly to the polymer, rather than being dispersed by “loading” into the polymer without chemical attachment, using any of several methods well known in the art and as described hereinbelow. The amount of bioactive agent is generally approximately 0.1% to about 60% (w/w) bioactive agent to polymer composition, more preferably about 1% to about 25% (w/w) bioactive agent, and even more preferably about 2% to about 20% (w/w) bioactive agent. The percentage of bioactive agent will depend on the desired dose and the condition being treated, as discussed in more detail below.

In addition to serving as stand-alone delivery systems for bioactive agents when directly administered in vivo, for example, in the form of inhalants, implants or local or systemic injectables, the invention AABB-PDP compositions can be used in the fabrication of various types of surgical devices. In this embodiment, the invention provides surgical devices comprising the invention AABB-PDP composition in which at least one bioactive agent is disbursed. Such surgical devices include solid implants, particles, and coatings of the composition on at least a portion of the surface of a surgical device. The AABB-PDP composition of which the surgical device is comprised will biodegrade so as to deliver to surrounding tissue in a controlled manner the bioactive agent(s) released from the polymer's backbone and/or dispersed in the polymer.

In one embodiment, the invention AABB-PDP composition can be fabricated in the form of a pad, sheet or wrap of any desired surface area. For example, the polymer can be woven or formed as a thin sheet of randomly oriented fibers. Such pads, sheets and wraps can be used in a number of types of wound dressings for treatment of a variety of conditions, for example by promoting endogenous healing processes at a wound site. The polymer compositions in the wound dressing biodegrade over time, releasing a disbursed bioactive agent, including a backbone therapeutic diol or di-acid, to be absorbed into a target cell in a wound site where it acts intracellularly, either within the cytosol, the nucleus, or both, or the bioactive agent can bind to a cell surface receptor molecule to elicit a cellular response without entering the cell. Alternatively, a bioactive agent released from the polymer composition, for example when used as the covering for a bioactive stent, promotes endogenous healing processes at the wound site by contact with the surroundings into which the wound dressing or implant is placed.

Bioactive agents contemplated for dispersion within the polymers used in the invention AABB-PDP compositions include anti-proliferants, rapamycin and any of its analogs or derivatives, paclitaxel or any of its taxene analogs or derivatives, everolimus, sirolimus, tacrolimus, or any of its -limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), cilostazol, and the like.

Suitable bioactive agents for dispersion in the invention AABB-PDP compositions and particles made therefrom also can be selected from those that promote endogenous production of a therapeutic natural wound healing agent, such as nitric oxide, which is endogenously produced by endothelial cells. Alternatively the bioactive agents released from the polymers during degradation may be directly active in promoting natural wound healing processes by endothelial cells. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenalin; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

A variety of bioactive agents, coating molecules and ligands for bioactive agents can be attached, for example covalently, to the surface of the polymer coatings or particles. Bioactive agents, such as targeting antibodies, polypeptides (e.g., antigens) and drugs can be covalently conjugated to the surface of the polymer coatings or particles. In addition, coating molecules, such as polyethylene glycol (PEG) as a ligand for attachment of antibodies or polypeptides or phosphatidylcholine (PC) as a means of blocking attachment sites on the surface of the particles, can be surface-conjugated to the particles to prevent the particles from sticking to non-target biological molecules and surfaces in a subject to which the particles are administered.

For example, small proteinaceous motifs, such as the B domain of bacterial Protein

A and the functionally equivalent region of Protein G are known to bind to, and thereby capture, antibody molecules by the Fc region. Such proteinaceous motifs can be attached as bioactive agents to the invention AABB-PDP compositions, especially to the surface of the polymer particles described herein. Such molecules will act, for example, as ligands to attach antibodies for use as targeting ligands or to capture antibodies to hold precursor cells or capture cells out of the blood stream. Therefore, the antibody types that can be attached to polymer coatings using a Protein A or Protein G functional region are those that contain an Fc region. The capture antibodies will in turn bind to and hold precursor cells, such as progenitor cells, near the polymer surface while the precursor cells, which are preferably bathed in a growth medium within the polymer, secrete various factors and interact with other cells of the subject. In addition, one or more bioactive agents dispersed in the polymer particles, such as the bradykinins, may activate the precursor cells.

In addition, bioactive agents for attaching precursor cells or for capturing progenitor endothelial cells (PECs) from a blood stream in a subject to which the polymer compositions are administered are monoclonal antibodies directed against a known precursor cell surface marker. For example, complementary determinants (CDs) that have been reported to decorate the surface of endothelial cells include CD31, CD34, CD102, CD105, CD106, CD109, CDw130, CD141, CD142, CD143, CD144, CDw145, CD146, CD147, and CD166. These cell surface markers can be of varying specificity and the degree of specificity for a particular cell/developmental type/stage is in many cases not fully characterized. In addition, these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood in the site into which the polymer particles are implanted for local delivery of the active agents. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies, and active fragments thereof.

The following bioactive agents and small molecule drugs will be particularly effective for dispersion within the invention AABB-PDP compositions, whether sized to form a time release biodegradable polymer depot for local delivery of the bioactive agents, or sized for entry into systemic circulation, as described herein. The bioactive agents that are dispersed in the invention AABB-PDP compositions and methods of use will be selected for their suitable therapeutic or palliative effect in treatment of a disease of interest, or symptoms thereof, or in experiments designed for in vitro testing of such effects in cells or tissue culture, or in vivo.

In one embodiment, the suitable bioactive agents are not limited to, but include, various classes of compounds that facilitate or contribute to wound healing when presented in a time-release fashion. Such bioactive agents include wound-healing cells, including certain precursor cells, which can be protected and delivered by the biodegradable polymer in the invention compositions. Such wound healing cells include, for example, pericytes and endothelial cells, as well as inflammatory healing cells. To recruit such cells to the site of a polymer depot in vivo, the invention AABB-PDP compositions and particles thereof used in the invention and methods of use can include ligands for such cells, such as antibodies and smaller molecule ligands, that specifically bind to “cellular adhesion molecules” (CAMs). Exemplary ligands for wound healing cells include those that specifically bind to Intercellular adhesion molecules (ICAMs), such as ICAM-1 (CD54 antigen); ICAM-2 (CD102 antigen); ICAM-3 (CD50 antigen); ICAM-4 (CD242 antigen); and ICAM-5; Vascular cell adhesion molecules (VCAMs), such as VCAM-1 (CD106 antigen); Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.

In another aspect, the suitable bioactive agents include extra cellular matrix proteins, macromolecules that can be dispersed into the polymer particles used in the invention AABB-PDP compositions, e.g., attached either covalently or non-covalently. Examples of useful extra-cellular matrix proteins include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human, but biocompatible, glycoproteins, such as alginates and chitin derivatives. Wound healing peptides that are specific fragments of such extra-cellular matrix proteins and/or their bio-mimics can also be used.

Proteinaceous growth factors are another category of bioactive agents suitable for dispersion in the invention AABB-PDP compositions and methods of use described herein. Such bioactive agents are effective in promoting wound healing and other disease states as is known in the art, for example, Platelet Derived Growth Factor-BB (PDGF-BB), Tumor Necrosis Factor-α (TNF-α), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art.

Alternatively, expression systems comprising vectors, particularly adenovirus vectors, incorporating genes encoding a variety of biomolecules can be dispersed in the invention AABB-PDP compositions and particles thereof for timed release delivery. Methods of preparing such expression systems and vectors are well known in the art. For example, proteinaceous growth factors can be dispersed into the invention AABB-PDP compositions for administration of the growth factors either to a desired body site for local delivery, by selection of particles sized to form a polymer depot, or systemically, by selection of particles of a size that will enter the circulation. Growth factors, such as VEGFs, PDGFs, FGF, NGF, and evolutionary and functionally related biologics, and angiogenic enzymes, such as thrombin, may also be used as bioactive agents in the invention compositions.

Drugs, either synthetically or naturally synthesized, are yet another category of bioactive agents suitable for dispersion in the invention AABB-PDP compositions and methods of use described herein. Such drugs include, for example, antimicrobials and anti-inflammatory agents as well as certain healing promoters, such as, for example, vitamin A and synthetic inhibitors of lipid peroxidation.

A variety of antibiotics can be dispersed as bioactive agents in the invention AABB-PDP compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc. (1996) 118: 13107-13108; J. Amer. Chem. Soc. (1997) 119:12041-12047; and J. Amer. Chem. Soc. (1994) 116:4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, including alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art.

Anti-inflammatory bioactive agents are also useful for dispersion in invention AABB-PDP compositions. Depending on the body site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin and mucous membrane agents. See, Physician's Desk Reference, 2005 Edition. Specifically, the anti-inflammatory agent can include dexamethasone, which is chemically designated as (11θ, 16I)-9-fluoro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The polypeptide bioactive agents included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS, p. 392; and Evans et al. (1987) J. Med. Chem., 30:1229) and are usually developed with the aid of computerized molecular modeling. Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al, Endocrinology (2003) 144:3262-3269,

It is readily apparent that the subject invention can be used to prevent or treat a wide variety of diseases or symptoms thereof.

Following preparation of the invention AABB-PDP compositions and polymer particles thereof, optionally loaded with at least one bioactive agent, the composition can be lyophilized and the dried composition suspended in an appropriate media prior to administration.

Any suitable and effective amount of the at least one bioactive agent can be released with time from the AABB-PDP composition, including those in a polymer coating on a medical device, such as a stent, an intraocular disc for implant or a depot formed from particles thereof introduced in vivo. The suitable and effective amount of the bioactive agent will typically depend, e.g., on the specific AABB-PDP polymer and concentration of therapeutic backbone diol or di-acid incorporated therein, type of particle or polymer/bioactive agent linkage, if present. Typically, up to about 100% of the backbone diol(s) or di-acid(s) and optional bioactive agent(s) can be released from polymer particles sized to avoid circulation as described herein that form a polymer depot in vivo. Specifically, up to about 90%, up to 75%, up to 50%, or up to 25% thereof can be released from the polymer depot. Factors that typically affect the release rate from the polymer depot are the nature and amount of the polymer/backbone therapeutic agent, the types of polymer/bioactive agent linkage, and the nature and amount of additional substances present in the formulation.

Once the invention AABB-PDP composition is made, as above, the composition is formulated for subsequent administration. Any suitable route of administration can be used depending of the formulation used, for example, by intrapulmonary, gastroenteral, subcutaneous, intramuscular, into the central nervous system, intraperitoneum or intraorgan delivery. For injection or inhalation, the compositions will generally include one or more “pharmaceutically acceptable excipients or vehicles” appropriate for oral, mucosal or subcutaneous delivery, such as water, saline, glycerol, polyethylene glycol, hyaluronic acid, ethanol, etc. Additionally, auxiliary substances, such as wetting or emulsifying agents, pH buffering substances, flavorings, and the like, may be present in such vehicles.

For example, intranasal and pulmonary formulations will usually include vehicles that neither cause irritation to the nasal mucosa nor significantly disturb ciliary function. Diluents such as water, aqueous saline or other known substances can be employed with the subject invention. The intrapulmonary formulations may also contain preservatives such as, but not limited to, chlorobutanol and benzalkonium chloride. A surfactant may be present to enhance absorption by the nasal mucosa.

For rectal and urethral suppositories, the vehicle composition will include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the invention AABB-PDP compositions can be formulated in pessary bases, such as those including mixtures of polyethylene triglycerides, or suspended in oils such as corn oil or sesame oil, optionally containing colloidal silica. See, e.g., Richardson et al., Int. J. Pharm. (1995) 115:9-15.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular invention AABB-PDP or particles thereof and mode of administration.

Alternatively, the invention AABB-PDP compositions can be formulated as coatings on medical devices for delivery of a bioactive agent to an in vivo site of implant. For example, the composition can be used to coat at least a portion of the surface of a vascular stent or an intraocular disc for rapid delivery of a bioactive agent, as described herein, to surrounding tissues or cells. Methods for making and using intraocular devices comprising polymers of the PEA family of polymers (e.g., invention AABB-PDP polymers and compositions either in the form of solid discs or as coatings on such discs), for delivery of opthalmologic agents are as disclosed in U.S. application No. 20070292476.

In addition to humans, the invention AABB-PDP compositions are also intended as delivery vehicles for use in veterinary administration of bioactive agents to a variety of mammalian patients, such as pets (for example, cats, dogs, rabbits, and ferrets), farm animals (for example, swine, horses, mules, dairy and meat cattle) and race horses.

In one embodiment, the AABB-PDP compositions used in the invention will comprise an “effective amount” of one or more backbone therapeutic diol or di-acid(s) and/or dispersed bioactive agents of interest. That is, an amount of such an agent will be incorporated into the composition that will produce a sufficient therapeutic or palliative response in order to prevent, reduce or eliminate symptoms. The exact amount necessary will vary, depending on the subject to which the composition is being administered, the age and general condition of the subject; the capacity of the subject's immune system, the degree of therapeutic or palliative response desired; the severity of the condition being treated or investigated; the particular bioactive agent(s) selected and mode of administration of the composition, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the active agent delivered per dose.

Once formulated, the invention AABB-PDP compositions can be administered in a variety of ways. In one embodiment, a suspension of molecules or particles is administered orally, mucosally, or by subcutaneously or intramuscular injection, and the like, using standard techniques. See, e.g., Remington: The Science and Practice of Pharmacy, Mack Publishing Company, Easton, Pa., 19th edition, 1995, for mucosal delivery techniques, including intranasal, pulmonary, vaginal and rectal techniques, as well as European Publication No. 517,565 and Illum et al., J Controlled Rel. (1994) 29:133-141, for techniques of intranasal administration. Surgical devices comprising AABB-PDP compositions containing one or more bioactive agents can be formulated as implantable solids, such as, for example, arterial stents or intraocular discs, or coatings on such surgical devices. Such implantables are surgically inserted using techniques well known in the art.

Dosage treatment may be a single dose of the invention AABB-PDP composition, or a multiple dose schedule as is known in the art. The dosage regimen, at least in part, will also be determined by the need of the subject and be dependent on the judgment of the practitioner. Furthermore, if prevention of disease is desired, the AABB-PDP composition (in the form of particles, or not) is generally administered prior to primary disease manifestation, or symptoms of the disease of interest. If treatment is desired, e.g., the reduction of symptoms or recurrences, the AABB-PDP compositions are generally administered subsequent to primary disease manifestation.

The formulations can be tested in vivo in a number of animal models developed for the study of oral, subcutaneous or mucosal delivery. For example, the conscious sheep model is an art-recognized model for testing nasal delivery of substances See, e.g., Longenecker et al., J. Pharm. Sci. (1987) 76:351-355 and Illum et al., J. Controlled Rel. (1994) 29:133-141. The AABB-PDP composition, generally in powdered, lyophilized form, is blown into the nasal cavity. Blood samples can be assayed for bioactive agent using standard techniques, as known in the art.

In another embodiment the invention provides surgical devices comprising the invention AABB-PDP polymer.

In still another embodiment, the invention provides methods for preparing an O,O′-diacyl-bis-(alpha hydroxy acid) (Compounds 1 herein) having a chemical formula described by structural formula (III)

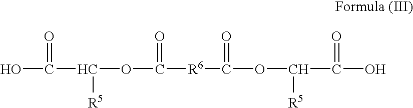

wherein R⁵ is H or —CH₃ and R⁶ is an acyl independently selected from (C₂-C₁₂) alkylene and (C₂-C₁₂) alkenylene, said method comprising:

a) forming an acid di-chloride of the acyl in a solvent that acts as a catalyst and hydrogen chloride acceptor;

b) interacting the acid di-chloride with glycolic or lactic acid in dry ethyl acetate in the presence of the solvent to form solid O,O′-diacyl-bis-(alpha hydroxy acid) product; and

c) collecting solid O,O′-diacyl-bis-(alpha hydroxy acid) product formed in a) from the solution.

Examples of suitable solvents for use in the invention methods for preparing an O,O′-diacyl-bis-(alpha hydroxy acid) include, for example, pyridine and triethylamine. The solid product can then be collected by filtering, for example on a porous glass filter, and washing with an aliquot of pH 2-3 water, acidified with hydrochloric acid. The filtrate will contain the major amount of the desired product. An additional amount of product may be collected from aqueous wash by extracting it with 3-4 portions, for example of about 100 mL each, of ethyl acetate. These ethyl acetate fractions can then be combined, dried, filtered and evaporated to dryness, resulting in further yield of intended product, with a yield of raw O,O′-adipoyl-bis-(glycolic acid) as great as about 70%. For purification, the product can be recrystallized from ethyl acetate/hexane 70/30 (v/v) mixture. If the product is based on sebacic acid, the product is not soluble in water and can be washed with water.

Reaction scheme I below illustrates this method for synthesizing compounds of Formula (III), wherein R⁶═(CH₂)₄ and (CH₂)₈:

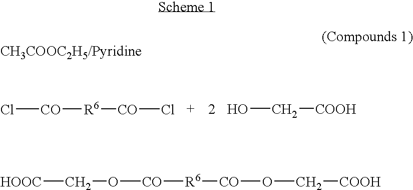

The di-acids of Compounds I are transformed into the active di-p-nitrophenyl esters thereof (Compounds 2 herein). An exemplary process for such transformation is shown in Scheme 2 below and further described in Example I herein:

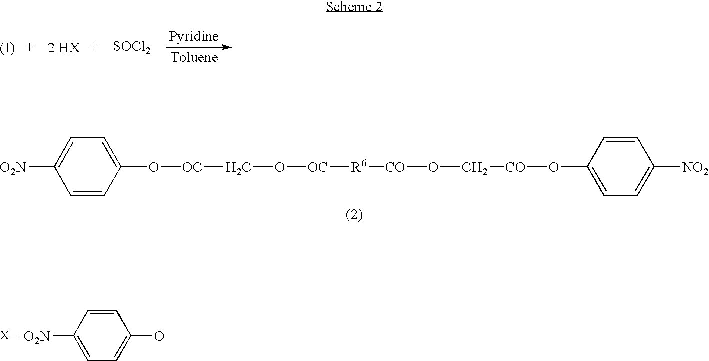

Synthesis of p-toluenesulfonic acid salts of bis(alpha-amino acid) diesters (Compounds 3) is well known in the art. Such synthesis is described, for example by R. Katsarava et al. (J. Polym. Sci, Part A: Polym. Chem. (1999) 37:391-407), and is further described in scheme 3 below and in Example I herein:

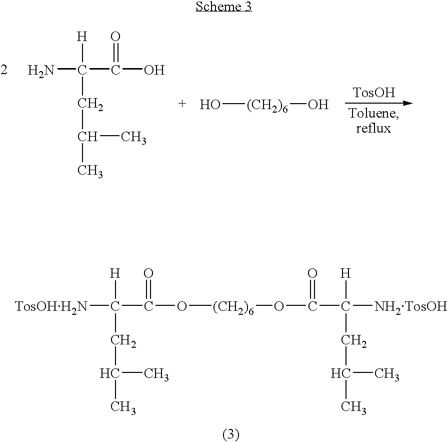

Syntheses of AA-BB type poly(depsipeptides) (AABB-PDPs) were carried out under the conditions of solution active polycondensation (APC) adapting a procedure reported previously (Katsarava et al., 1999 supra) in N,N-dimethylacetamide, using triethylamine as an acid acceptor. Active diesters (Compounds 2) were reacted with amino acid derived monomers—di-p-toluenesulfonic acid salts of bis-α-amino acid)-α,ω-alkylene diesters (Compounds 3)—according to Scheme 4 below and yielded compounds of general structural formula I.1:

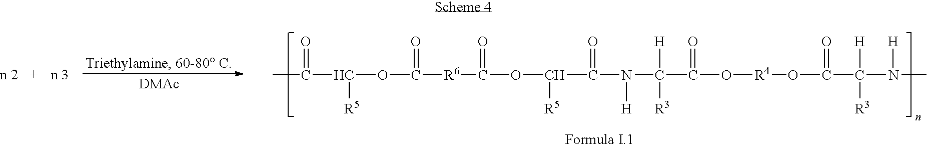

The invention is further illustrated by the following Examples, which are intended to illustrate and not to limit the invention.

### Example 1

### Monomer Syntheses

A. Synthesis of O,O′-diacyl-bis-(glycolic acid)s (Compounds 1)

Starting monomers for new AABB_polydepsipeptides-O,O′-diacyl-bis-(glycolic acid)s of general structure (1) were synthesized by interaction of diacid chlorides with glycolic acid in dry ethyl acetate in the presence of pyridine as a catalyst preparing an O,O′-diacyl-bis-(alpha hydroxy acid) and HCl acceptor, according to Scheme 1 herein.

Synthesis of O,O′-adipoyl-bis-(glycolic acid), (Compound 1.1): 15.82 g (0.2 mol) of glycolic acid was dissolved in 500 mL of dry ethyl acetate and then about 400 mL of the solvent was distilled off to remove water that was present in commercial glycolic acid. To the residual solution 23.9 g (0.1 mol) of adipoyl chloride was added, chilled to 0° C. and 16.3 mL (0.2 mol) of pyridine solution in 50 mL of the same solvent was added drop-wise on stirring. After pyridine addition was complete the reaction mixture was stirred at room temperature for an additional 2 h. The solid product was filtered off on a porous glass filter and washed with 200 mL, pH 2-3 water, acidified with hydrochloric acid. The filtrate contained the major amount of the desired product. An additional amount of product was collected from aqueous wash by extracting it with 3-4 portions, of about 100 mL each of ethyl acetate. These ethyl acetate fractions were then combined, dried over Na₂SO₄, filtered and evaporated to dryness, resulting further yield of intended product. Total yield of raw O,O′-adipoyl-bis-(glycolic acid) (compound 1.1, R⁶═(CH₂)₄), was 70%. For purification, the product was recrystallized from ethyl acetate/hexane 70/30 (v/v) mixture. The yield of the purified product was 50-55%, m.p. 98-100° C. Acid number: calculated 262, found 262; Elemental analysis C₁₀H₁₄O₈ (262.21): calculated C 45.81; H 5.38; found C 45.67; H 5.12.

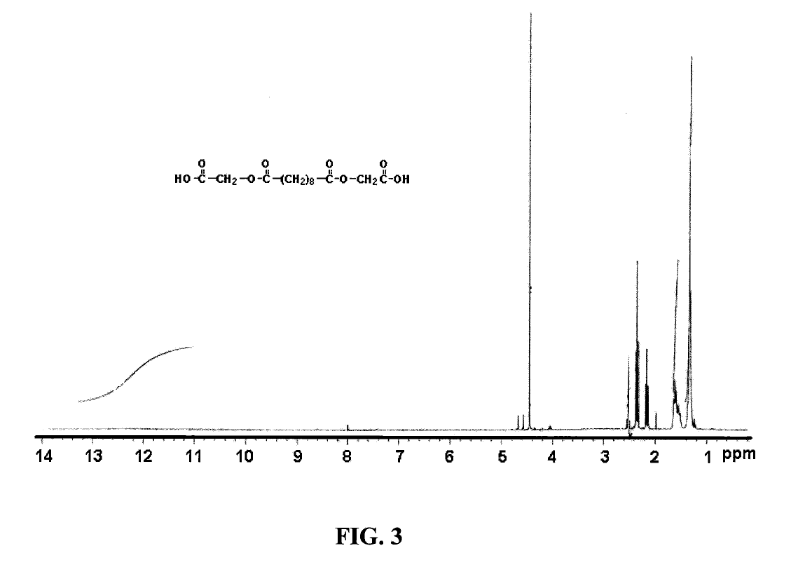

A scan of the FTIR spectrum of compound 1.1 is shown in FIG. 2. A wide carbonyl absorption band at 1727 cm⁻¹ could be ascribed to both ester and COOH carbonyls. The ¹H NMR spectrum, a scan of which is shown in FIG. 3, provided data in accordance with this assumed structure.

Synthesis of O,O′-sebacoyl-bis-(glycolic acid), (compound 1.2, R⁶═(CH₂)₈): Synthesis was carried out using a process analogous to that used for the adipic acid derivative, (Compound 1.1) above. Solid waste product formed was filtered off and washed with pH 2-3 water, acidified with hydrochloric acid, then with distilled water and dried under reduced pressure at 50° C. No additional portion of the product was obtained from the filtrate. (It has to be noted that the diester-diacid (Compound 1.2), being based on the more hydrophobic sebacic acid, is not soluble in water and can be washed with water).

Yield of raw O,O′-sebacoyl-bis-(glycolic acid) (compound 1.2) was 70%. The product was recrystallized from ethylacetate/hexane 70/30 (v/v) mixture. The yield of the purified product was 50-55%, m.p. 121° C.-123° C. Acid number: calculated 352, found 352; Elemental analysis, C₁₄H₂₂O₈ (314.32): calculated C 52.83; H 6.97; found C 52.67; H 7.12. The FTIR spectrum of compound 1.2, wherein R⁶═(CH₂)₈, showed two carbonyl absorption bands at 1727 cm⁻¹ (carboxyl CO) and 1757 cm⁻¹ (ester CO), confirming the assumed structure.

B. Synthesis of di-p-nitrophenyl esters of O,O′-diacyl-bis-(glycolic acid)ₛ, (Compounds 2).

The di-acids of Compounds 1 were transformed into the active di-p-nitrophenyl esters of Compounds 2 using a process shown in Scheme 2 herein.

Synthesis of active diester of O,O′-adipoyl-bis-(glycolic acid) (Compound 2.1, R⁶═(CH₂)₄): In 250 mL of dry toluene, 26.2 g (0.1 mole) of O,O′-adipoyl-bis-(glycolic acid) (compound 1.1), 27.8 g (0.2 mole) of p-nitrophenol and 32.5 mL of pyridine were suspended and chilled to 0-5° C. A solution of 14.5 g (0.2 mole) of thionyl chloride in 50 mL of dry toluene was added drop-wise to the reaction mixture and the temperature was kept at <5° C. in an ice bath. After complete addition of the thionyl chloride, the ice bath was removed and the mixture was stirred at room temperature for an additional 2 h. White solid formed was filtered off, washed with acidified water (HCl, pH 3-4) and dried in vacuum at 40° C.-45° C. in the presence of phosphorus pentoxide. The obtained active diester (compound 2.1) was recrystallized from ethylacetate/chlorobenzene 50/50 (v/v) mixture to yield 60% product with m.p.=162° C.-164° C. Elemental analysis: Calculated for C₂₂H₂₀N₂O₁₂ (504.4), C, 52.39; H, 4.00; N, 5.55; Found: C, 52.48; H, 3.93; N, 5.64.

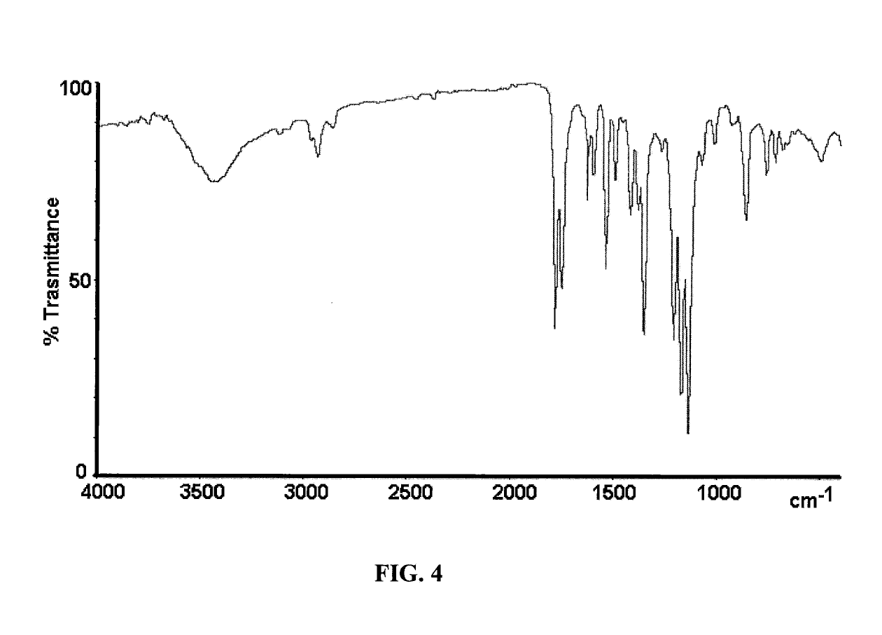

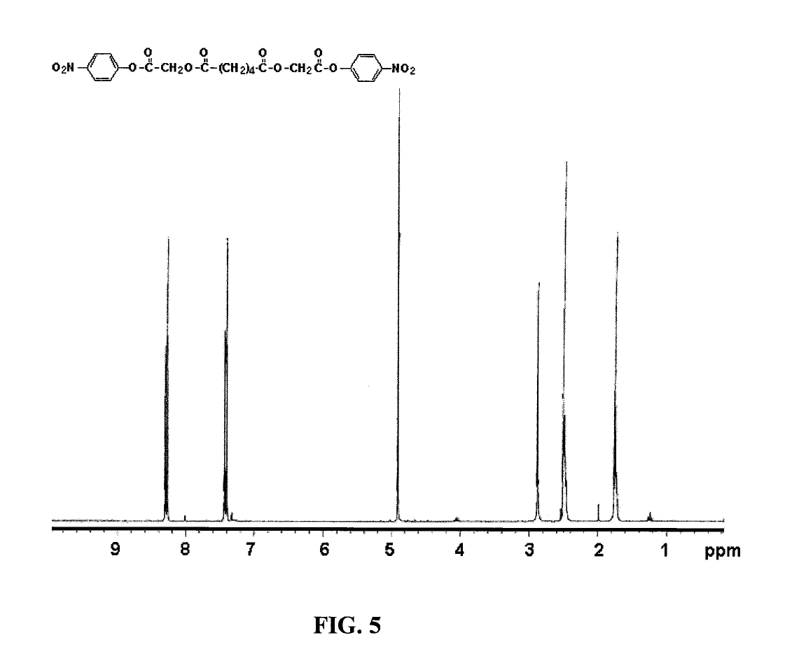

An FTIR spectrum (in Nujol) of the diester (compound 2.1) confirmed the assumed structure, by showing an absorption band at 1774 cm⁻¹ reflecting an active ester bond between glycolic acid and p-nitrophenol, and at 1743 cm⁻¹ reflecting regular ester bonds between adipic and glycolic acids (See FIG. 4). The ¹H NMR spectrum of compound 2.1 (FIG. 5) was in accordance with this assumed structure as well.

Synthesis of active diester (Compound 2.2 (R⁶═(CH₂)₈):

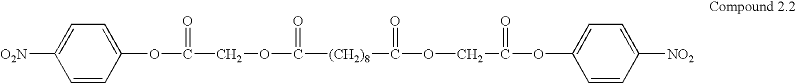

This synthetic reaction was carried out using a procedure similar to that used for synthesis of the active derivative of adipic acid (Compound 2.1), except that after thionyl chloride was completely added, the reaction mixture was stirred for 0.5 h at ambient temperature, then at 60° C. until complete dissolution of the solid products. Upon refrigeration overnight, precipitate was formed overnight, was then filtered off, washed with acidified water (HCl, pH 3-4) and dried in vacuum at 40° C.-45° C. using phosphorus pentoxide. Yield of raw active ester (Compound 2.2) was 61%, with m.p. 75° C.-80° C. After repeated (5 times) recrystallizations from ethylacetate/n-hexane 70/30 (v/v) mixture, the melting point was increased to 100° C.-101.5° C. The data of elemental analysis confirmed the structure of the active diester (Compound 2.2): Calculated for C₂₆H₂₈N₂O₁₂, (560.4): C, 55.71; H, 5.04; N, 5.00; Found: C, 55.58; H, 5.23; N, 5.14.

The FTIR spectrum of the obtained product, Compound 2.2, also confirmed the assumed structure—as expected two absorption bands were observed in the spectrum of this compound: one of them at 1735 cm⁻¹ reflects an ester bond between sebacic and glycolic acids, and another one at 1774 cm⁻¹ reflects the “active” ester bond between glycolic acid and p-nitrophenol.

Synthesis of Compound 2.2 in chlorobenzene: Initially this reaction was carried out in toluene as described above. However, chlorobenzene was found to be a better solvent for this reaction; yield of raw product was thereby increased up to 75% and m.p. was raised to 84° C.-90° C. (vs. 75° C.-80° C. when synthesized in toluene). A desirable m.p. of 100° C.-101.5° C. was achieved after double recrystallization of product from an ethylacetate/n-hexane (70/30 (v/v)) mixture.

C. Synthesis of p-toluenesulfonic acid salts of bis(alpha-amino acid) diesters (Compounds 3)

Synthesis was carried out according to the previously published procedure (Katsarava R, et al. J. Polym. Sci, Part A: Polym. Chem. (1999) 37:391-407), as shown in scheme 3 herein.

Into 250 mL of toluene in a flask equipped a Dean-Stark apparatus and overhead stirrer, were placed L-Leucine (0.132 mol), p-toluenesulfonic acid monohydrate (0.132 mol) and 1,6-hexanediol (0.06 mol). The heterogeneous reaction mixture was heated to reflux for about 12 h until 4.3 mL (0.24 mol) of water evolved. The reaction mixture was then cooled to room temperature, filtered, washed with acetone, and recrystallized twice from methanol/toluene 2:1 (v/v) mixture. Yields as well as melting points were identical to published data.

### D. Polymer Syntheses.

Syntheses of AA-BB type poly(depsipeptides) (AABB-PDPs) were carried out under the conditions of solution active polycondensation (APC), adapting a procedure reported previously (Katsarava et al., 1999 supra) in N,N-dimethylacetamide, using triethylamine as an acid acceptor. Active diesters (Compounds 2) were reacted with amino acid derived monomers—di-p-toluenesulfonic acid salts of bis-α-amino acid)-α,ω-alkylene diesters (Compounds 3)—according to Scheme 4 below and yielded compounds of general structural formula I.1:

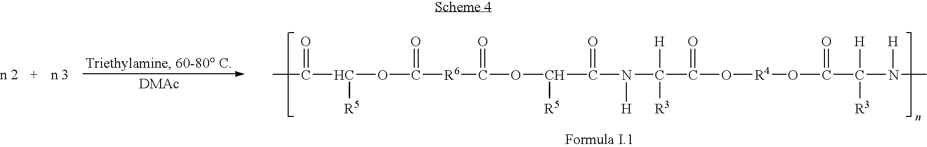

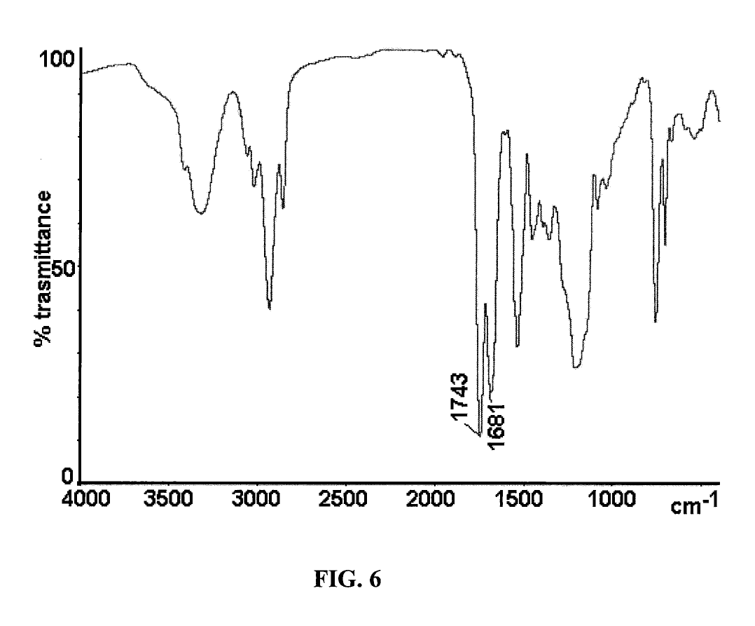

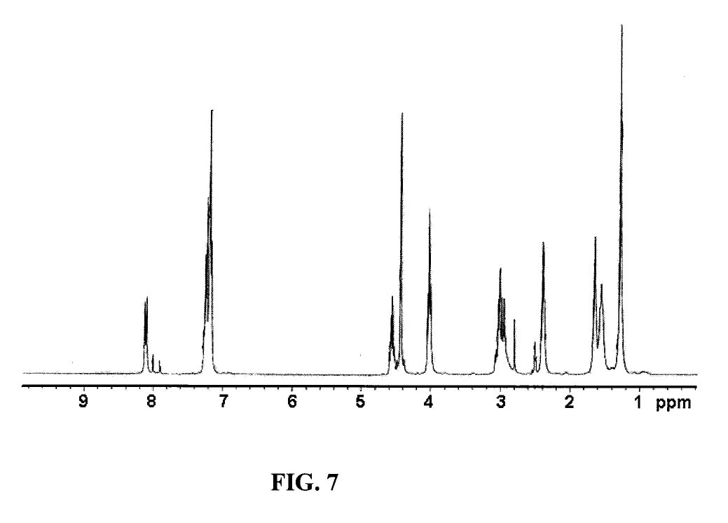

High-molecular weight AABB-PDPs were synthesized in N,N-dimethylacetamide (DMAc) via solution Active Polycondensation of (Compounds 2) with diesters (Compounds 3), which were based on L-leucine, L-phenylalanine and aliphatic diols, specifically Leu-6, Leu-8, Leu-12, Phe-6 and Phe-8. M_{w} of the PDPs ranged from 35,000 to 46,000; M_{w}/Mₙ ranged from 1.36 to 1.46). The structures of AABB-PDPs for selected samples were confirmed by FTIR (FIG. 6), and by ¹H NMR (FIG. 7) and ¹³C NMR, as well as by elemental analysis data.

### Example 2

To study whether formed polymer AABB-PDPs will undergo hydrolysis in water a series of tests were conducted. Initially, the diamine monomer (Compound 3) selected for use was the one with the longest aliphatic chain—Leu-12, based on L-leucine and 1,12-dodecane diol. During the work-up a portion of the reaction solution was precipitated and washed with water and dried; while another portion was precipitated in ethanol, washed with ethanol and dried. In a third experiment, dry polymer was kept in water for 48 h, and then dried. Molecular weights of the various polymers prepared by these methods were estimated by GPC in 0.1 N LiBr/DMF using PS standards. The results of these water hydrolysis tests are summarized in Table 1 below.

TABLE 1

| # | PDP 4-GA-Leu-12* | Mw | Mn | Mw/Mn |
|---|---|---|---|---|
| 1 | Separated in water and washed with | 36,000 | 25,200 | 1.43 |
| | water | | | |
| 2 | Separated in ethanol and | 46,000 | 32,800 | 1.40 |
| | washed with ethanol | | | |
| 3 | #1 above washed with ethanol (a low- | | | |
| | molecular-weight fraction dissolved in | | | |
| | ethanol): | | | |
| 3a | High-molecular-weight fraction: | 45,000 | 34,000 | 1.32 |
| 3b | Low-molecular-weight fraction: | 18,000 | 15,000 | 1.20 |
| 4 | #2 above placed in distilled | 46,000 | 33,000 | 1.39 |
| | water at r.t. for 48 h. | | | |

*AABB-PDP prepared from adipic acid = (4), sebacic acid = (8), glycolic acid = (GA), L-leucine = (Leu) and 1,12-dodecanediol = (12).

The results of the water hydrolysis tests showed that sample #1, which was separated in water, had M_{w}=36,000 Da, and sample #2, which was separated in ethanol, had a M_{w}=46,000 Da. The lower M, of sample #1 can be attributed to the presence of low-molecular-weight fractions (sample #3b) that were removed after washing sample #1 with ethanol. The M_{w} of the high-molecular-weight sample #3a that remained after washing with ethanol had the same M_{w} as sample #2, which was obtained after separation of the polymer in ethanol. When later placed in water at room temperature for 48 h, sample #2 retained its molecular weight and polydispersity (as shown by sample #4). These experiments illustrate that no substantial biodegradation of the invention PDP took place after its contact with water at room temperature, indicating that the invention AABB-PDPs are rather stable in water under neutral conditions. This discovery allowed for separation of the ethanol-soluble invention polymers (AABB-PDPs containing short chain leucine-based monomers—Leu-6 and Leu-8) in water. AABB-PDPs based on containing phenylalanine were separated in ethanol.

The M_{w} characteristics of synthesized AABB-PDPs described by general formula (I) are compiled in Table 2 below.

TABLE 2

| # | PDP | Mw | Mn | Mw/Mn |
|---|---|---|---|---|
| 1 | 4-GA-Leu-6 | 38,000 | 26,600 | 1.43 |
| 2 | 4-GA-Leu-8 | 44,800 | 30,600 | 1.46 |
| 3 | 4-GA-Leu-12 | 46,000 | 32,800 | 1.40 |
| 4 | 4-GA-Phe-6 | 38,700 | 27,400 | 1.41 |
| 5 | 4-GA-Phe-8 | 35,000 | 25,600 | 1.36 |
| 6 | 8-GA-Leu-6 | 38,800 | 27,000 | 1.44 |
| 7 | 8-GA-Leu-8 | 47,500 | 33,500 | 1.42 |
| 8 | 8-GA-Leu-12 | 52,500 | 35,500 | 1.48 |
| 9 | 8-GA-Phe-6 | 32,000 | 21,900 | 1.46 |

¹⁾GPC experiments were carried out in N,N-diemthylformamide (PS standards)
Designations: The acid moiety in invention PDPs are designated as;
4-GA- for PDPs based on adipic acid (compound 2.1)
8-GA- for PDPs based on sebacic acid (compound 2.2)

The structures of invention AABB-PDPs (for selected samples 8-GA-Phe-6 and 8-GA-Leu-12) were confirmed by FTIR (FIG. 6). The 8-GA series AABB-PDPs were sent for elemental and NMR analysis. All the polymers listed in Table 2 showed good film-forming properties.

Systematic studies of the physical-chemical, mechanical, and in vitro biodegradation properties of invention AABB-PDPs are in progress.

### Example 3

### Thermal Properties of Invention AABB-PDPs

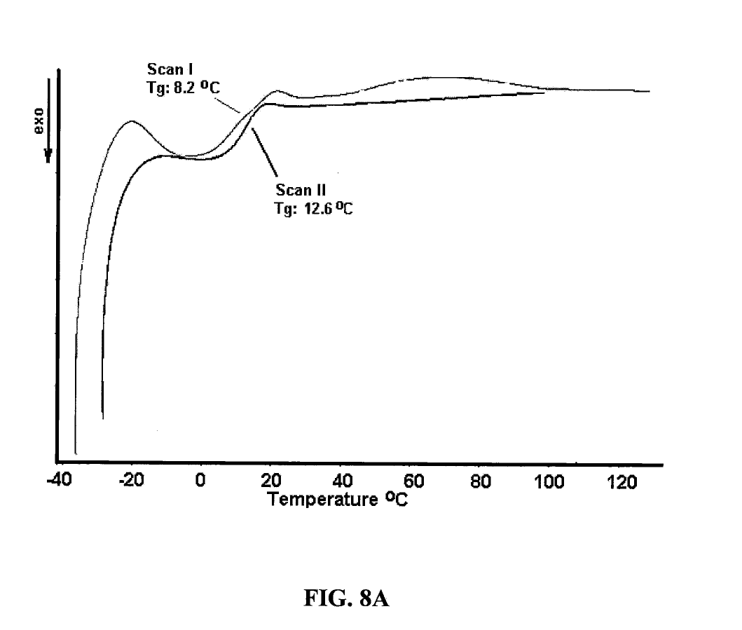

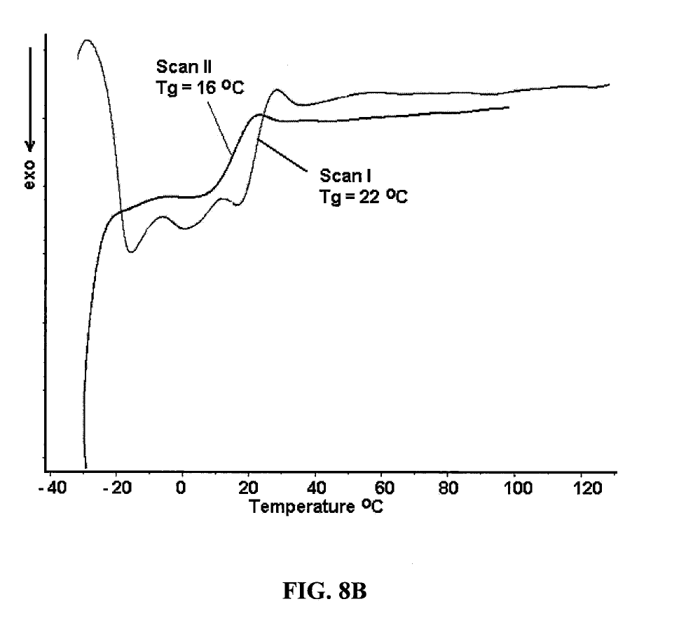

Thermograms of two selected samples of invention AABB-PDPs were conducted at a heating rate 10° C./min under N₂ as shown in FIGS. 8A and B. The glass transition temperature (T_{g}) of the sample 4-GA-Leu-12 lies within the range of 8° C.-13° C. (data from two scans). No crystalline phase was observed in these scanned polymers. A very wide endotherm occurring in the range of 50° C.-100° C. (FIG. 8A) could be ascribed to the melting of hydrophobic domains formed by the long hydrophobic 1,12-dodecamethylene chain of the diol residue. By contrast, the T_{g} of polymer 4-GA-Phe-8 (FIG. 8B) lies in the range of 16° C.-22° C. (data of two scans) and is somewhat higher than that of 4-GA-Leu-12. This result is expected and can be attributed to the presence of a shorter polymethylene chain of the 1,8-octanediol residue and higher macrochain rigidity, hence, higher T_{g}, of Phe-based PEAs as a group. A very weak and wide endotherm in the region 40° C.-60° C. (FIG. 8B) could be ascribed to extremely weak hydrophobic interaction of the 4-GA-Phe-8 molecules.

### Example 4

### In Vitro Biodegradation Study of AABB-PDPs

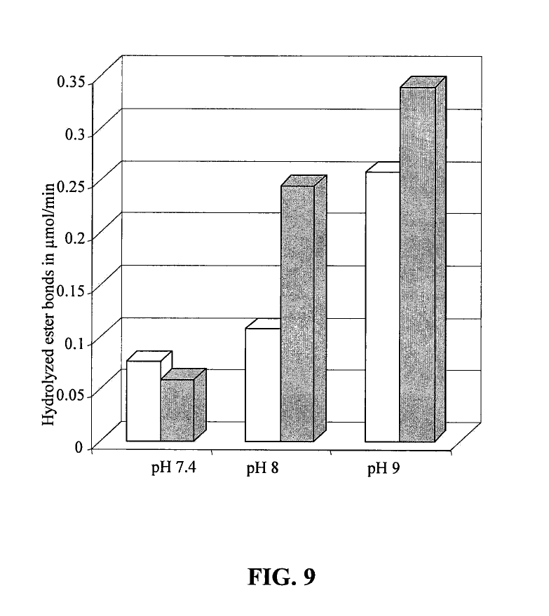

In vitro non-specific biodegradation of invention AABB-PDPs was assessed at various Ph values using potentiometric titration (FIG. 9). AABB-PDP 4-GA-Leu-12 was used in this study, and compared with the hydrolysis rate of regular PEA 8-Leu-6 (wherein in Formula I, R¹ would be ═(CH₂)₈, R³═(CH₂CH(CH₃)₂), R⁴═(CH₂)₆). An automatic potentiometric titrator (Metrohm-842 Titrando) and 0.02 N NaOH water solution were used to determine the hydrolysis rates at Ph values 7.4, 8 and 9, which rates were assessed in μmole of NaOH consumed during 1 min (tmole/min) and correspond to the quantity of ester bonds cleaved during 1 min in each polymer.

The results summarized in FIG. 9 show that the hydrolysis rate of PDP 4-GA-Leu-12 at alkaline pH (8 and 9) is higher than the hydrolysis rate of PEA 8-Leu-6, as was expected. A systematic study of non-specific (chemical) and lipase catalyzed in vitro hydrolysis of PDP and PEAs of related structures is in progress now.

Preliminary results in such studies show a high rate of nonspecific chemical hydrolysis due to the presence in the invention polymers of polarized ester bonds formed by glycolic acid residues (for the preliminary results see below). This characteristic is believed to be important for biodegradation of devices implanted in in vivo body sites where the concentration of bioenzymes (e.g., proteases and esterases) is negligible.

An increased rate of lipase-catalyzed biodegradation is expected due to increased concentration of ester bonds in polymeric backbones of AABB-PDPs. This result is expected to enhance in vivo biodegradation of devices made using the invention polymers and which are destined for applications in contact with the blood stream (e.g., arterial stents) where the concentration of lipase and related enzymes is lower than optimal for enzymatic cleavage.

Biodegradation of invention AABB-PDPs is expected to form readily digestible fragments at a rate more rapid than that of PEAs. After cleavage (hydrolysis) of the polymeric backbones of regular PEAs, the initial breakdown products are diols and N,N′-diacyl-bis-α-amino acids (Compound 1.VII), which contains amide linkages and can be digested to ultimate products under the action of another class of enzymes—acylases (whose catalytic scission of amide bonds is much more rapid than chemical (nonspecific) hydrolysis of amide bonds. By contrast, biodegradation of an invention AABB-PDP, as illustrated by Compound 1.VIII herein, contains easily cleaved ester bonds, forming readily digestible breakdown products: 2 moles of depsipeptide (Compound 1.IX below) and one mole of diacid:

HO—CH₂—CO—NH—CH₂—COOH and HOCO—(CH₂)_{y}—COOH (Compound 1.Ix)

Thus, invention AABB-PDPs can be considered more digestible and more rapidly biodegraded than regular PEAs.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A composition comprising a AABB-polydepsipeptide (AABB-PDP) having a chemical formula described by general structural formula (I), 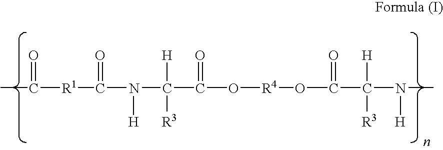
wherein n ranges from about 5 to about 150;
wherein at least one R¹ is independently selected from residues of O,O′-diacyl-bis-(alpha hydroxy acid) of formula (III) below, wherein in Formula (III) R⁵ is H or methyl and R⁶ is independently (C₂-C₁₂) alkylene or (C₂-C₁₂) alkenylene, and additional R¹s can be selected from the group consisting of (C₂-C₂₀) alkylene, (C₂-C₂₀) alkenylene, α,ω-bis(4-carboxyphenoxy)-(C₁-C₈) alkane, saturated or unsaturated residues of therapeutic di-acids, and combinations thereof;
R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), saturated or unsaturated therapeutic diol residues, and combinations thereof; 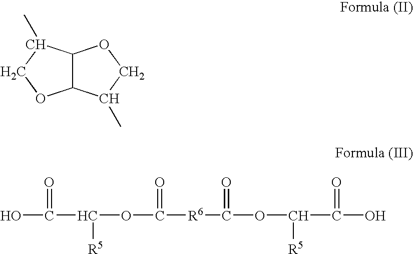
or a AABB-PDP having a chemical formula described by structural formula (IV): 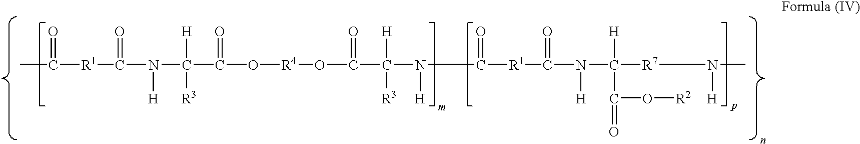
wherein n ranges from about 5 to about 150, m ranges about 0.1 to 0.9; p ranges from about 0.9 to 0.1;
at least one R¹ is independently selected from residues of O,O′-diacyl-bis-(alpha hydroxy acid) of formula (III) below, wherein R⁵ is H or methyl and R⁶ is independently selected from (C₂-C₁₂) alkylene and (C₂-C₁₂) alkenylene, and additional R's can be selected from the group consisting of (C₂-C₂₀) alkylene, (C₂-C₂₀) alkenylene, α,ω-bis(4-carboxyphenoxy)-(C₁-C₈) alkane, saturated or unsaturated residues of therapeutic di-acids, and combinations thereof;
R² is independently selected from the group consisting of hydrogen, (C₁-C₁₂) alkyl, (C₆-C₁₀) aryl or a protecting group;
R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₂₀) alkylene, (C₂-C₂₀) alkenylene, (C₂-C₈) alkyloxy, (C₂-C₂₀) alkylene, bicyclic-fragments of 1,4:3,6-dianhydrohexitols of structural formula (II), residues of saturated or unsaturated therapeutic diols and combinations thereof; and
R⁷ is independently selected from the group consisting of (C₂-C₂₀) alkyl and (C₂-C₂₀) alkenyl.

2. The composition of claim 1, wherein in formula (III), R⁵ is H.

3. The composition of claim 1, wherein in formula (III) R⁵ is CH₃.

4. The composition of claim 1, wherein R⁶ is independently selected from the group consisting of (CH₂)₄, (CH₂)₆, and (CH₂)₈.

5. The composition of claim 1, wherein R⁷ in formula (IV) is independently selected from the group consisting of (C₃-C₆) alkyl and (C₃-C₆) alkenyl.

6. The composition of claim 1, wherein at least one R¹ is an α,ω-bis(4-carboxyphenoxy)-(C₁-C₈) alkane.

7. The composition of claim 1, wherein at least one R⁴ is a saturated or unsaturated residue of a therapeutic diol.

8. The composition of claim 1, wherein the composition further comprises dispersed therein at least one bioactive agent, which composition biodegrades to release the bioactive agent over a period of from about one week to about six months.

9. The composition of claim 8, wherein the composition is formulated for administration in the form of a liquid dispersion of molecules or polymer particles.

10. The composition of claim 9, wherein in the particles have an average diameter in the range from about 10 nanometers to about 1000 microns.

11. The composition of claim 8, wherein the composition is formulated as a wound dressing.

12. The composition of claim 8, wherein the composition is formulated as an implantable surgical device or as a coating on at least a surface portion of a surgical device.

13. The composition of claim 12, wherein the composition is formulated as an intraocular implant.

14. The composition of claim 1, wherein the AABB-PDP has an average molecular weight in range from about 20,000 Da to about 80,000 Da.

15. A method for preparing an O,O′-diacyl-bis-(alpha hydroxy acid) having a chemical formula described by structural formula (III) 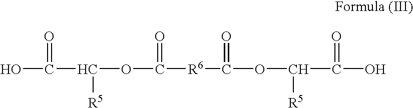
wherein R⁵ is H or —CH₃ and R⁶ is an acyl independently selected from (C₂-C₁₂) alkylene and (C₂-C₁₂) alkenylene, said method comprising:
a) forming an acid di-chloride of the acyl in an organic basic solvent that acts as a hydrogen chloride acceptor and catalyst;
b) interacting the acid di-chloride with glycolic or lactic acid in dry ethyl acetate in the presence of the solvent to form a solution containing solid O,O′-diacyl-bis-(alpha hydroxy acid) product; and
c) collecting the solid O,O′-diacyl-bis-(alpha hydroxy acid) product formed in b) from the solution.

16. The method of claim 15, wherein R⁵ is H and R⁶ is (CH₂)₄ or (CH₂)₈.

17. The method of claim 15, wherein R⁵ is —CH₃ and R⁶ is (CH₂)₄ or (CH₂)₈.

18. A method for delivering a biologic agent to a subject comprising administering to the subject a composition of claim 1 having a biologic agent dispersed therein so as to deliver the biologic agent to the subject in vivo.

19. The method of claim 18, wherein the composition is formulated as a liquid dispersion of molecules or injectable polymer particles and the administering is by injection.

20. The composition of claim 18, wherein the composition is formulated as a wound dressing and the bioactive agent promotes wound healing.

21. The composition of claim 18, wherein the composition is formulated as an implantable surgical device or as a coating on at least a surface portion of a surgical device and the administering is by surgical implanting.

22. The composition of claim 12, wherein the composition is formulated as an intraocular implant and the surgical implanting is intraocular.
